(19) United States
(12) Patent Application Publication
(10) Pub. No.: US 2010/0325769 A1
(43) Pub. Date: Dec. 30, 2010

(54) ARTICLE OF CLOTHING INCORPORATING A CONTRACEPTIVE DEVICE AND THE METHOD OF EFFECTUATING THE PROMOTION OF AN AGENDA USING THE SAME

(76) Inventor: Katy Tseng, Walnut, CA (US)

Correspondence Address: WPAT, PC;INTELLECTUAL PROPERTY ATTORNEYS, 2030 MAIN STREET, SUITE 1300, IRVINE, CA, 92614, US

(21) Appl. No.: 12/492,525

(22) Filed: Jun. 26, 2009

(51) Int. Cl. A41D 1/00; A41D 27/08; A41D 27/20; A61F 6/04

(52) U.S. Cl. 2/69; 2/244; 2/247; 128/844

(57) ABSTRACT

A condom-incorporated garment and a method of interactive advertisement using the garment to promote a marketing, educational or social agenda, where the condom-incorporated garment has a piece of garment with pocket, a colored condom in a sealed package placed in the pocket. The color condom is shown through a window and overlays on top of a graphical or textual message. The graphical or textual message can be highlighted by the color condom. Alternatively, the graphical or textual message can be hidden from plain view by the color condom. The pocket and the color condom are part of the overall graphic design of the garment, allowing the wearer or a third party to remove the condom, thus transpiring a different message.

### CROSS-REFERENCE TO RELATED APPLICATIONS

Not Applicable.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC

Not Applicable.

### BACKGROUND OF THE INVENTION

(1) Field of the Invention

The field of the invention is garment and articles of clothing, more specifically, an article of clothing incorporating a contraceptive device to effectuate the promotion of an agenda.

(2) Description of Related Art Including Information Disclosed Under 37 CFR 1.97 and 1.98

Garments are known for centuries to have pockets so that a wearer of the garment may use the pocket to carry items, and to keep such items away from public viewing.

While some pockets are hidden and undetectable from public viewing, for example, disposed on the inside of the garment or disposed between layers of the garment, other pockets are sewn on the outside of the garment, and may be there for the same purpose of storing items or merely for aesthetic purposes. Despite the numerous ways to utilize a pocket, known pockets are merely for storage and/or aesthetic functions.

Contraceptive devices such as condoms are portable and can be placed inside the pocket of a garment. Typically, condoms can be carried in a pocket hidden from public view for fear of embarrassment. Also, it is undesirable to carry condoms and expose condoms in plain view, because condom manufacturers have warned consumers against exposing condoms to direct sun light or extreme temperatures.

Therefore, there is no known method to incorporate contraceptive devices into a garment such that the contraceptive device is shown to the public in plain view. There is a continuing need for new ways to incorporate contraceptive devices in a garment to effectuate the promotion of a marketing agenda, a social agenda, an educational agenda, or a design agenda.

All referenced patents, applications and literatures are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply. The invention may seek to satisfy one or more of the above-mentioned desires. Although the present invention may obviate one or more of the above-mentioned desires, it should be understood that some aspects of the invention might not necessarily obviate them.

### BRIEF SUMMARY OF THE INVENTION

Among the many different possibilities contemplated, the contraceptive device-incorporated garment has a garment with a contraceptive device coupled to it. Contemplated garment can include, but not limited to, a T-shirt, a button-up shirt, a pair of pants, an undershirt, a pair of underpants, pajama, lingerie, a jacket, a skirt, a scarf, a sock, and a hat.

In preferred embodiments, the contraceptive device is a packaged condom, more preferably, a colored condom in a sealed package where at least one side of the package is transparent so the color of the condom is visible by the viewing public.

Further, it is contemplated that the contraceptive device is coupled to the garment via a pocket. In other words, the garment has a pocket to house the contraceptive device. In one embodiment, the pocket is attached to the outside of the garment, and the pocket has a transparent window so that the contraceptive device is shown through the window. In yet another embodiment, the pocket is attached to the inside of the garment, and the garment has a transparent window so that the contraceptive device carried in the inside pocket is shown through the window. In still yet another embodiment, the pocket is disposed between layers of the garment, and the garment has a transparent window so that the contraceptive device carried in the pocket is shown through the window.

Contemplated window can have various sizes and shapes. Preferably the window is circular, or a generally circular shape. Among the many possible sizes contemplated for a circular window, it is preferred that the window has a diameter at and between 3.0 cm to 4.5 cm, more preferably, 3.5 cm to 4.0, even more preferably, 3.8 cm. In the alternative, the entire pocket can be transparent.

Contemplated pocket has a compartment, as all typical pockets do. The compartment is sized to desirably position the contraceptive device in relation to the window, so that the contraceptive device is strategically shown through the window. In one preferred embodiment, the compartment allows very little freedom of side-to-side movement of the contraceptive device.

Contemplated pockets can be attached to the garment by any and all known ways to provide a pocket to a garment, such as by sewing, or by using adhesives.

Another aspect of the contemplated inventive subject matter is methods to effectuate the promotion of a marketing agenda, a social agenda, an educational agenda, or a design agenda by incorporating contraceptive devices into a garment.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWINGS

FIG. 1A is a frontal view of a first embodiment of inventive subject matter, showing a T-shirt and a pocket yet to be attached to the T-shirt;

FIG. 1B is a frontal view of a first embodiment of inventive subject matter, showing a T-shirt and a pocket attached to the T-shirt;

FIG. 1C is a frontal view of a first embodiment of inventive subject matter, showing a packaged color condom in a pocket, highlighting an underlying graphic;

FIG. 1D is a frontal view of a first embodiment of inventive subject matter, showing a packaged color condom in a pocket, blocking an underlying graphic;

FIG. 2A is an illustration of another embodiment having a window of an inside pocket incorporated into a design pattern to deliver a message, wherein the pocket is empty, showing the underlying message;

FIG. 2B is an illustration of another embodiment having a window of an inside pocket incorporated into a design pattern to deliver a message, wherein a color condom is in the pocket, highlighting the underlying message;

FIG. 2C is an illustration of another embodiment having a window of an inside pocket incorporated into a design pattern to deliver a message, wherein a color condom is in the pocket, blocking the underlying message;

FIG. 3 illustrates various different types of garment contemplated;

FIG. 4 illustrates various different types of garment contemplated;

FIG. 5 illustrates various different types of garment contemplated;

FIG. 6 illustrates various different types of garment contemplated; and

FIG. 7 illustrates contemplated shapes of pockets.

### DETAILED DESCRIPTION OF THE INVENTION

It is important to appreciate that although garments are known to have pockets, some of which are capable of carrying a condom, it is considered undesirable to carry condoms in plain view because it attracts unwanted attention and causes embarrassment. Also, it is desirable to prevent exposure of condoms to sun light. The present invention is particularly innovative not only for its aesthetic function, but more for its function as part of an overall novel method of interactive advertising and message presentation

The invention and its various embodiments can now be better understood by turning to the following detailed description of the preferred embodiments, which are presented as illustrated examples of the invention defined in the claims. It is expressly understood that the invention as defined by the claims may be broader than the illustrated embodiments described below.

The words used in this specification to describe the inventive subject matter and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

As used herein, the term “garment” refers to an article of clothing that are made of any suitable material intended for that particular type of garment. The garment can include, but is not limited to a t-shirt, a button-up shirt, a pair of pants, an undershirt, a pair of underpants, pajama, lingerie, a jacket, a skirt, a scarf, a sock, and a hat.

As used herein, the term “contraceptive device” refers to birth control devices for male and/or female. The contemplated contraceptive device can include, but is not limited to, a condom.

The inventor has discovered an effective method of promoting marketing agenda, social agenda, educational agenda, or a design agenda by infusing messages of the agenda with a condom, and incorporating the infused message on a garment. In another embodiment, the method further includes infusing the message of the agenda with a condom, into a graphic display on a garment.

Condoms are known to carry certain levels of social stigma, and often create controversies among different social, religious, and cultural groups. Because of its controversial nature, physical incorporation of the actual condom onto a garment creates an immediate attention grabber, especially if the condom is in plain view of the public, and strategically positioned on the garment.

In one contemplated method, the condom-incorporated garment can be used to promote a marketing agenda. The marketing agenda can a part of a product marketing propaganda to emphasize certain products or events being advertised on the garment, by first directing a viewer's attention to the condom incorporated on the garment.

In yet another embodiment, the product marketing propaganda can be to encourage purchase of the garment by directing a purchaser's attention to the condom incorporated on the garment.

In another contemplated method, the condom-incorporated garment can be used to promote a social agenda. For example, the agenda can be a conservative social agenda on sexual health, or a liberal social agenda on sexual health. In some preferred embodiments, the method is not to merely attach a condom to a garment, but to strategically position a colored condom over a textual message printed on the garment. As such, the textual message on the garment is highlighted by and shown through the colored condom. In yet other embodiments, the method engages a wearer of the garment to selectively express these agenda by attaching or detaching a condom from the garment. When a condom is detached from the garment, an underlying graphic or textual display is revealed to the public, expressing the agenda.

In further contemplated methods, the condom-incorporated garment can be used to promote an educational agenda. The contemplated educational agenda can be for sexual health, sexual disease prevention, or for non-sexual related education agenda. For example, the agenda can be to teach people about saving the endangered sperm whale, or to teach people about the importance of recycling.

Various different methods and embodiments of promoting these agendas will be discussed below and by the accompanied drawing illustrations. Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, the embodiments are discussed in context of promoting a particular agenda, but one of ordinary skill in the art would immediate recognize that the embodiment can also be implemented to promote other agendas.

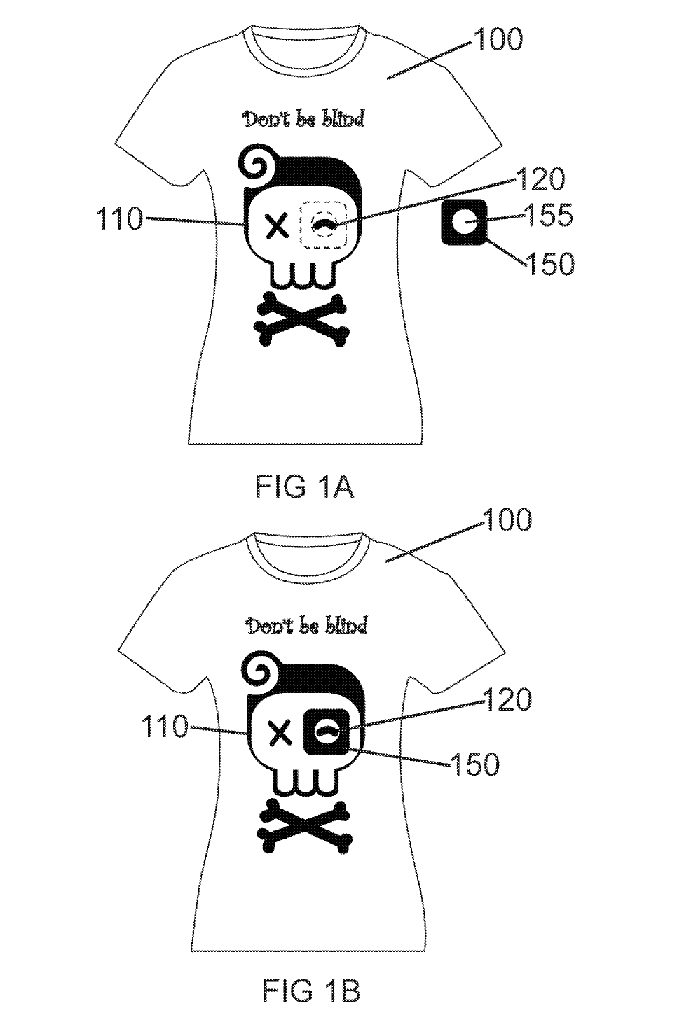

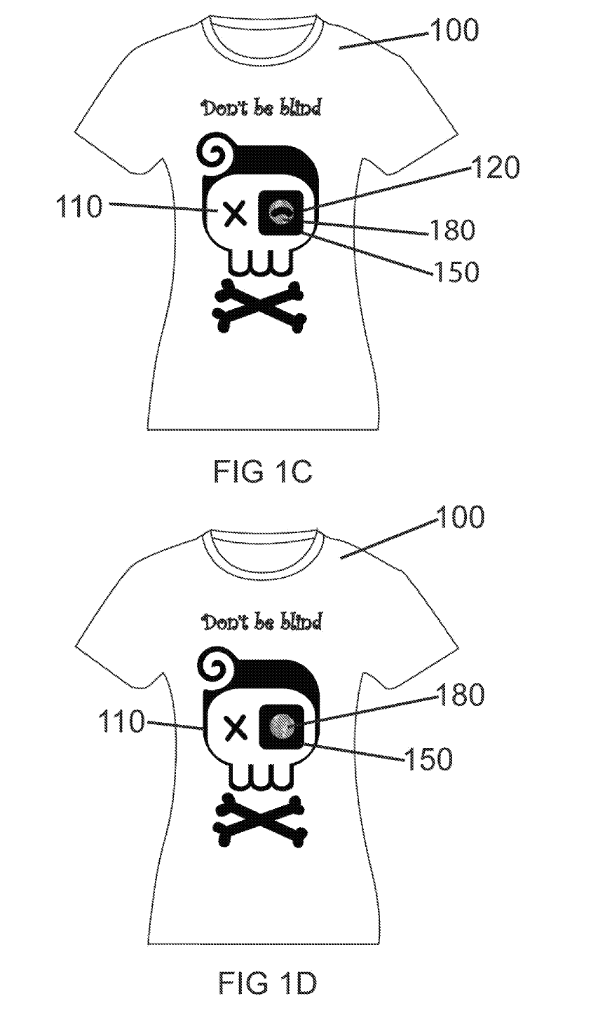

Referring now to FIGS. 1A-1D, a T-shirt 100 has a graphic design 110 on its front side. The graphic design 110 has an element 120 that is overlaid by a pocket 150. The importance of element 120 will be discussed in further detail below. In FIG. 1A, the pocket 150 is not yet attached to the T-shirt. The pocket 150 has a circular window 155. The window 155 can be an opening, allow anyone direct access to the interior compartment of the pocket through the window. Alternatively, the window preferably is made of a transparent material such as vinyl, so that the content of the pocket is in plain view of the public.

With respect to the outside pocket 150, it is attached to and disposed on the outside of the T-shirt 100, wherein the pocket 150 is attached to the T-shirt 100 by stitches, adhesives, or any other known methods of manufacturing a garment with pockets. Here, a square-shaped pocket is shown. Preferably, both lateral edges, and the bottom edge of the pocket 150 is sewn shut to the T-shirt 100, leaving only the top edge open for insertion of the condom 180. Alternatively, the pocket can be sewn to leave just one lateral edge, or both later edges open. One of ordinary skilled in the art would immediately recognize many other ways to attach the pocket 150 to the T-shirt 100 so as to leave at least one opening to insert a condom 180.

In a less preferred embodiment, the pocket 155 is sealed shut, leaving the condom 180 inside of the inner compartment of the pocket 155. To remove the condom 180, the consumer would have to partially remove the stitches from the pocket 150.

In FIG. 1B, the pocket 150 is attached to the outside of the T-shirt as an outside pocket. In FIG. 1B, a condom has yet to be inserted in the pocket 150. Element 120 of the graphic design 110 is now covered by the pocket 150, and shown through and encircled by the window 155. In this embodiment, element 120 creates part of the graphic design 110, and pocket 150 (along with window 155) also contributes to part of the graphic design 110. The combination of these creates a powerful tool to promote a sexual health message. Here, the overall graphic design includes a skull having an eye patch, eye expressions, and the slogan “Don't be blind.” The eye patch is the pocket 150, and when the condom is removed from the pocket 150, a smiling eye expression 120 is shown through the window 155, encouraging safe sexual practice by using condoms.

Referring now to FIG. 1C, a color condom 180 in a sealed package is placed in the pocket 150. The sealed package is contemplated to have two transparent sides so that the smiling eye expression 120 can be shown through the color condom. Here, smiling eye expression 120 is shown through the color condom 180 (represented by striations). This combination also creates a powerful “Don't′ be blind” message, using the color condom to highlight the smiling eye expression 120.

Referring now to FIG. 1D, a color condom 180 in a sealed package is placed in the pocket 150. The sealed package is contemplated to have only one transparent side so that the smiling eye expression 120 cannot be shown through the color condom. Here, smiling eye expression 120 is not shown through the color condom 180 (represented by striations). This combination of graphic design 110, slogan, pocket 150, window 155, and color condom 180 also creates a powerful “Don't′ be blind” message, using the color condom to block the smiling eye expression 120. Here, a viewer of the T-shirt would be tempted to help the skull face “see” better, by removing the condom 180 from the eye patch 150. Once the condom 180 is removed, the viewer is pleasantly surprised to find a smiling eye expression 120.

As illustrated by FIGS. 1A-1D also provides an interactive educational tool to drive the underlying agenda further. It is interactive in that a third party seeing someone wearing the T-shirt 100 would be tempted to manipulate the condom 180, thus revealing the message hidden under the condom. In FIGS. 1A-1D, the message is simply a graphical display 120. This graphical display 120 corresponds with the overall graphic design 110 to drive the intended agenda.

As illustrated in FIGS. 1B, 1C, and 1D, the same graphical elements 120 can transpire three different messages, depending on whether or not a condom is placed in the pocket, and if so, whether or that the underlying graphical element 120 can be shown through the color condom 180. In FIG. 1B, the overall message is a happy skull, not blinded by the eye patch. In FIG. 1C, the overall message is a happy skull, smiling through a condom, not blinded by the eye patch. In FIG. 1D, the overall message is an unhappy and intimidating skull face, blinded by the eye patch.

In some preferred embodiments, the graphic design 110 has a color that is as same as the color of the condom 180 as shown through the window 155. This is desirable to incorporate the condom 180 into the overall graphic design 110.

As for the size and shape of the window 155, FIGS. 1A-1D show a window 155 circular in shape. In one preferred embodiment, the circular window 155 has a diameter of between and including 3.0 cm to 4.5 cm, more preferably, 3.5 cm 4.0 cm, even more preferably, 3.5 cm to 3.8 cm, and most preferably, 3.8 cm.

In some embodiments, the window has a diameter of no greater than 3.5 cm. Because a typical condom has a rim of about or slightly smaller than 3.5 cm, these embodiments will minimize the chance of putting the rim of the condom in plain view of the public. This way, only the color of the condom 180 is shown in plain view, and the condom is not readily recognizable, making the garment more appropriate for a wearer who feels more comfortable being less provocative.

In other embodiments, the window has a diameter of greater than 3.5 cm. Because a typical condom has a rim of about or slightly smaller than 3.5 cm, these embodiments will likely show the rim of the condom 180 through window 155, appropriate for wearers who likes to make an aggressive statement promoting a particular agenda.

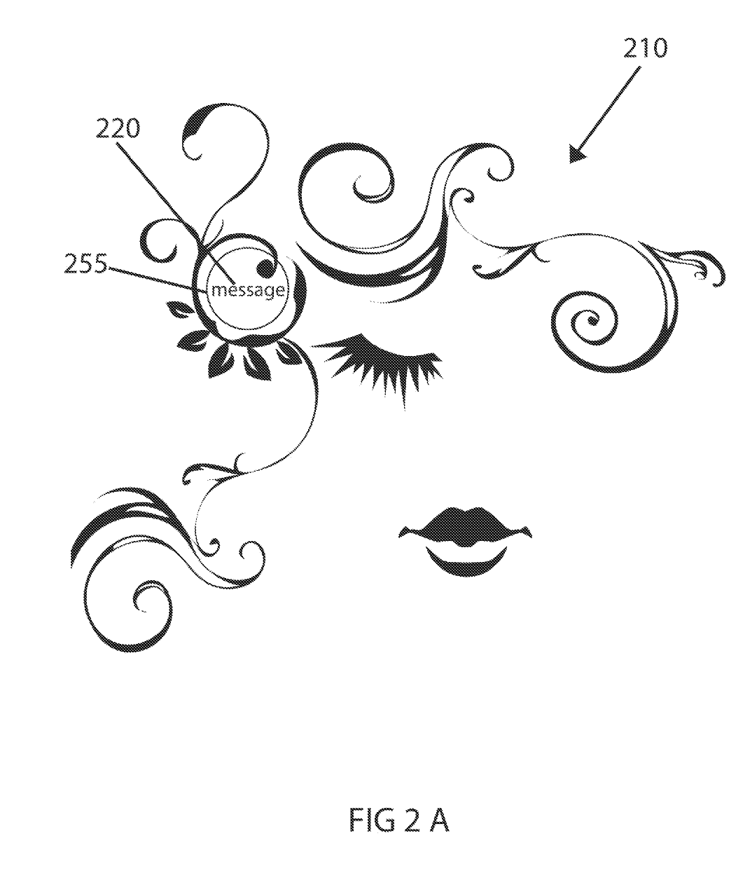

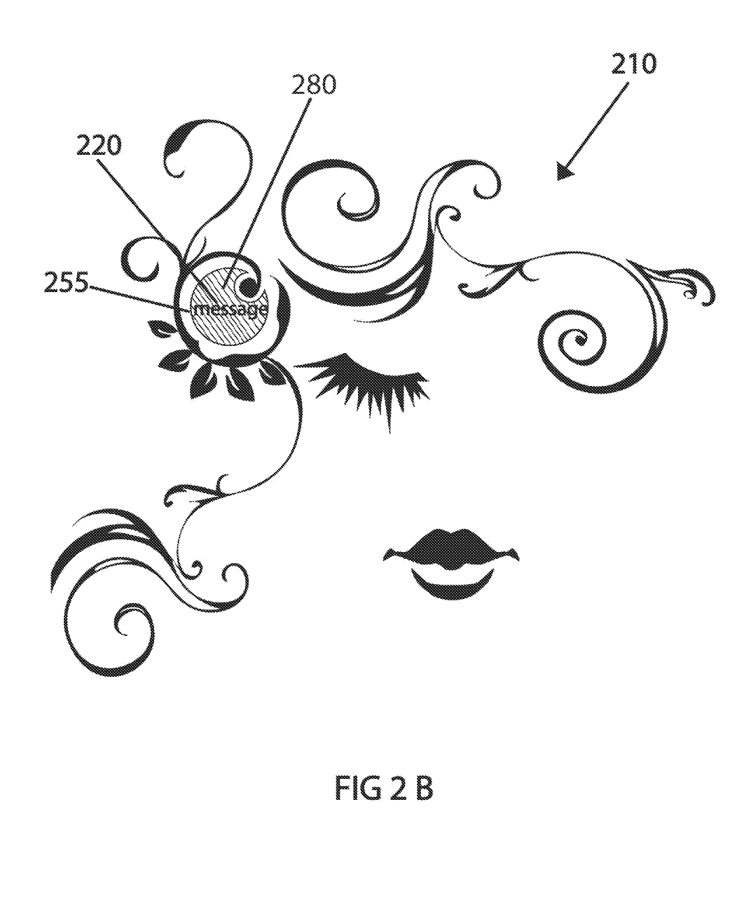

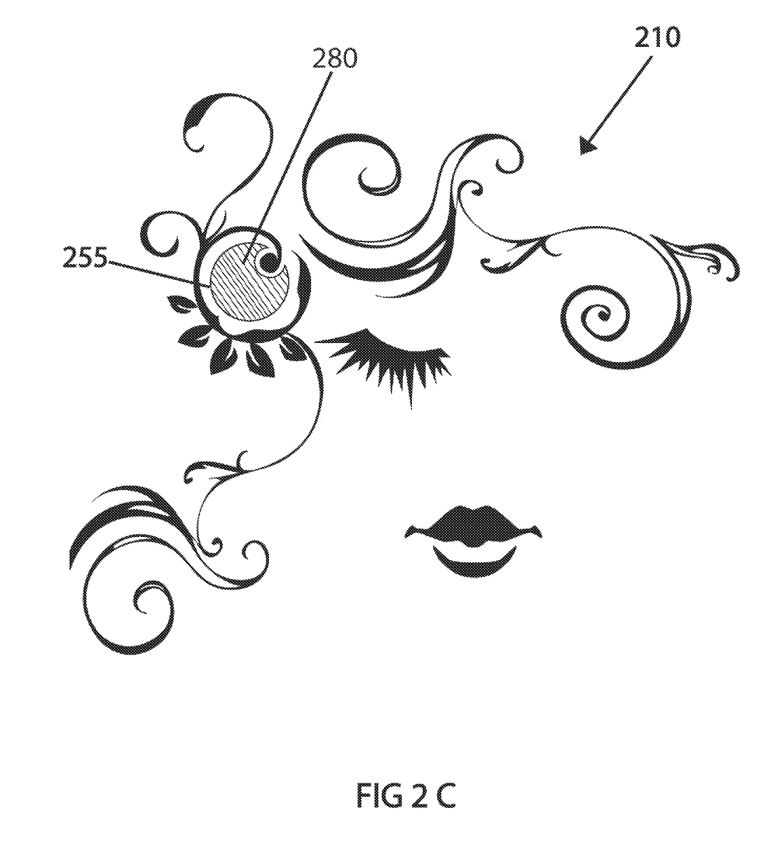

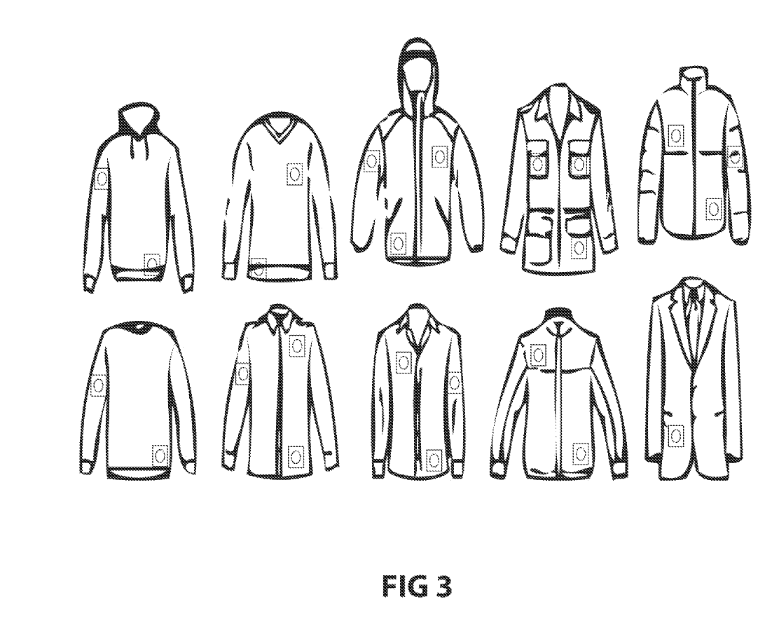

Referring now to FIGS. 2A-2C, illustrating embodiments where a textual message is used instead of a graphical element 120 of FIGS. 1A-1D. For ease of illustration, the garment is not shown in these drawing figures; one of ordinary skill in the art will immediately recognize that the embodiments shown here are suitable for all contemplated garment types. Here, FIG. 2A shows a graphic design 210 of a face with a window 255 incorporated into the design 210. The window appears to be a circular fruit attached to a vine. Here, an inside pocket (not shown) is used so that the pocket is not in plain view of the public. The window 255 is strategically positioned on the garment (not shown) so that a color condom 280 placed in the inside pocket can be shown through the window 255. As discussed previously, the window 255 can be an opening, allowing direct access of the inner compartment of the inside pocket from the outside. More preferably, the window 255 is a transparent vinyl.

In FIG. 2B, a color condom 280 is placed inside the pocket, and the color condom is shown through the window 255. Further, the color condom is in a seal package having two transparent sides, so that the underlying textual message 220 is in effect highlighted by the color condom 280 and shown through the color condom.

As previously discussed, the textual message can be pre-printed on the garment, and can contain meanings to promote a social, educational, political, or design agenda. For example, the textual message 220 can be a fun fact regarding the rain forest: “Although tropical rainforests cover just 2% of the Earth's land surface, they are home to two-thirds of all the living species on the planet.” Or, the textual message can be a slogan promoting safe sex: “pack it in plastic, it'll be fantastic!” or, “Don't′ be silly, protect your willie,” or “Don't′ be a fool, wrap your tool.”

In other embodiments, pre-printed cards can be provided so the consumer may insert cards of different messages to be shown through the window 225. Still further contemplated embodiments include blank cards for the consumer to write their own slogan.

Referring now to FIG. 2C, a color condom 280 is inserted into the pocket and shown through window 255. The condom 280, however, has a sealed package where one side is not transparent. As a result, the textual message 220 is blocked by the condom 280. In yet another embodiment, the graphic design 210 includes a pre-printed trivial question, and the answer to the question is the textual message 220 hidden behind the condom 280. Such embodiment creates an interactive educational tool to promote the intended agenda.

The contemplated pockets can have various different shapes and sizes. In all of the embodiments, the preferred pocket has an inner compartment sufficiently large to enclose a condom package, while giving the condom package very little free of movement, especially side-to-side movement. A typical condom package has the dimension of 6.0 cm by 6.0 cm. Preferred pocket has a square shape, as illustrated in FIGS. 1A-1D, and has an outside dimension of 7.0 cm by 7.0 cm. The pocket is attached to the garment in such ways as to create an inner compartment having a width of between 6.0 cm to 6.5 cm. This way, when the packaged condom is inserted from a top opening into the pocket's compartment, it has only about 0.5 cm of freedom of movement. This way, the color condom can be strategically shown through the window. As such, preferred window has a periphery wherein the periphery is about 1.0 cm to 1.5 cm away from an edge of inner compartment, because in a typical condom package, the rim of the condom is about 1.0 cm to 1.5 cm from the edge of the condom package.

In yet another embodiment, instead of having a transparent window, the entire pocket can be transparent, or be made of transparent or semi-transparent material. The transparent/semi-transparent material can be suitable materials to allow a view to visibly detect a condom and its color from the outside. Such materials include natural and synthetic polymers, naturally occurring materials, textile fibers, and all reasonable combinations thereof.

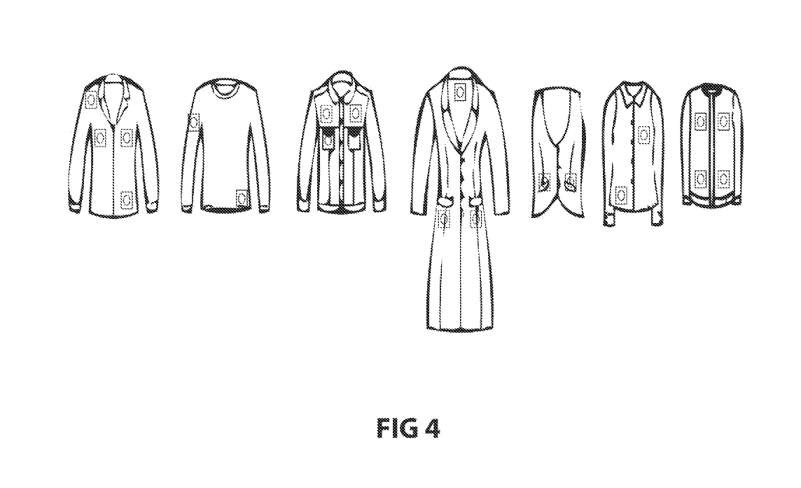

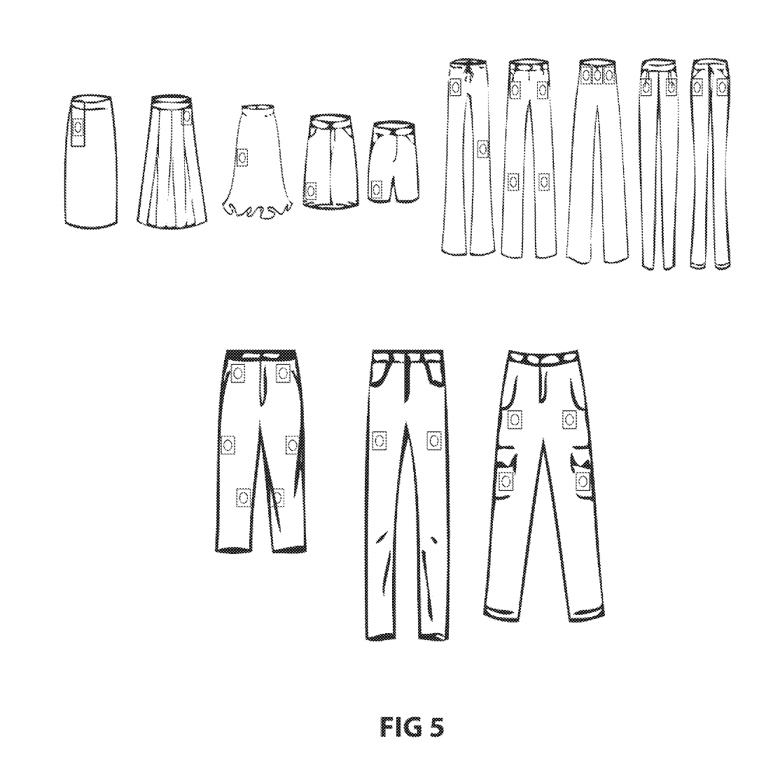

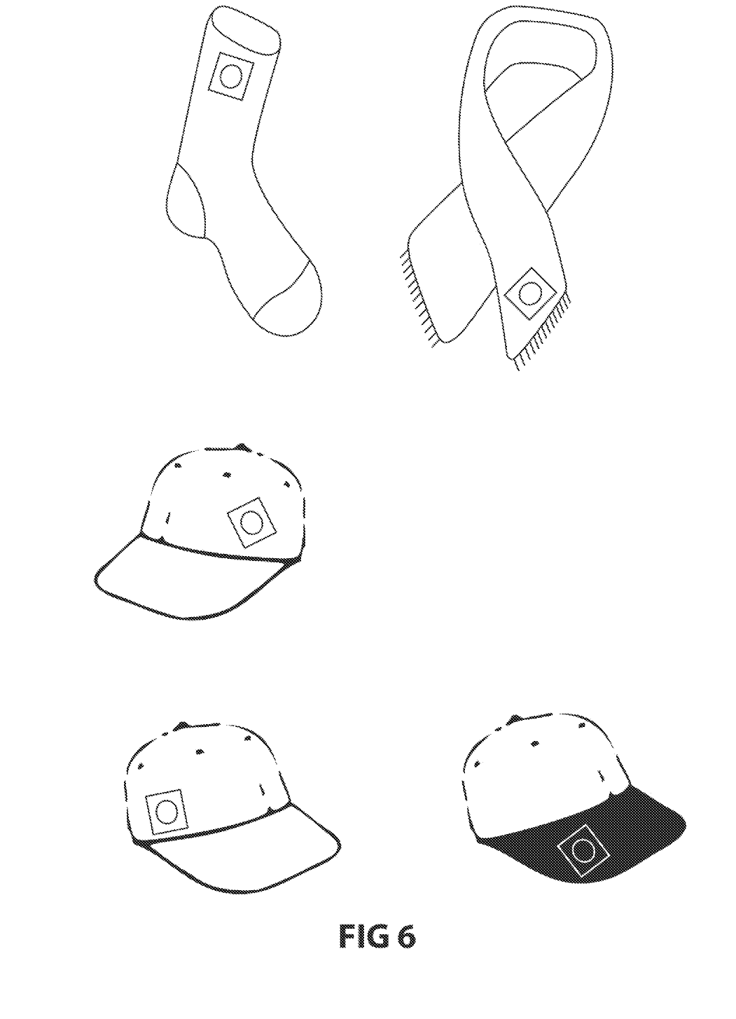

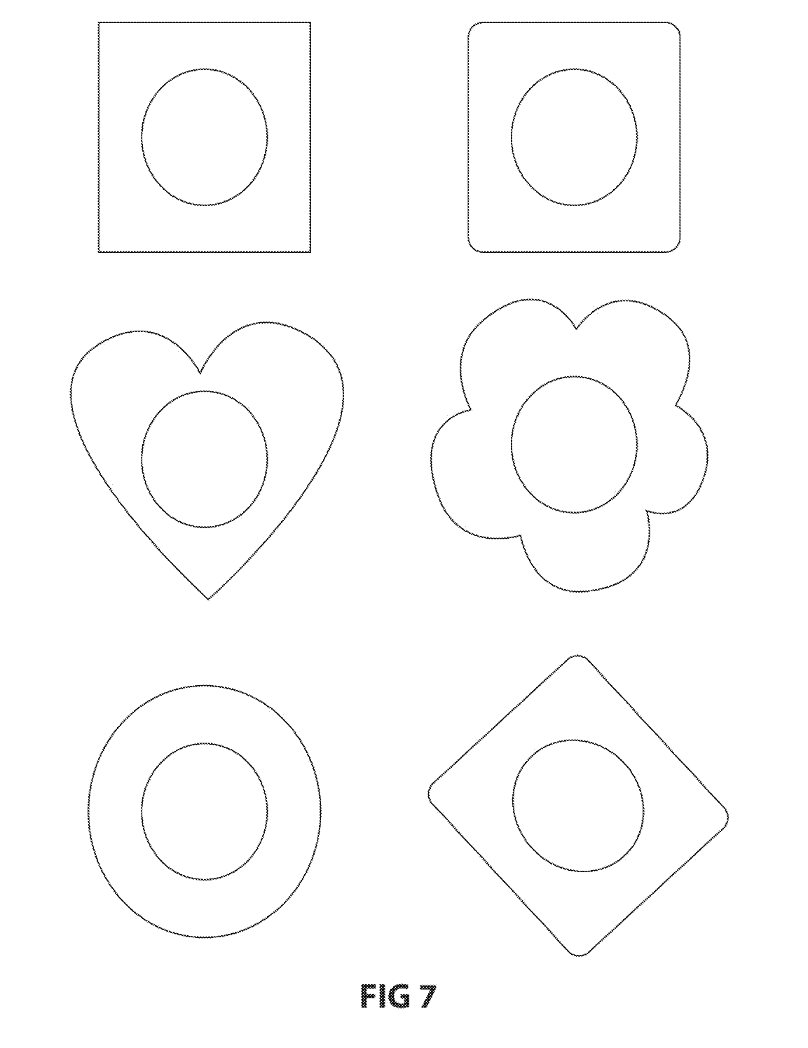

FIGS. 3-6 illustrate examples of garment that can be used in conjunction with the disclosed inventive subject matter. The preferred embodiments can have at least one pocket disposed on the garment, and can be located anywhere on the garment as needed as determined by the overall graphical and aesthetic design of the garment. Because these figures are used to show possible locations for pockets, graphical designs are not shown in these examples. It should be noted that in preferred embodiments of the invention, overall graphical design is an integral part to effectuate the promoting of the intended agenda. Contemplated garments include t-shirts, button-up shirts, sweat shirts, pants, jeans, khakis, shorts, undershirts, underpants, pajama, lingerie, jackets, coats, rain coats, skirts, suit jackets, sweater, scarves, socks, hats, and vests.

FIG. 7 illustrates contemplated shapes of the pockets. Shapes and sizes of the pockets can be varied to supplement the overall graphic design on the garment.

The definitions of the words or elements of the following claims include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Thus, specific embodiments and applications of Articles of Clothing Incorporating a Contraceptive Device and Method of Effectuating the Promotion of an Agenda Using the Same have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Insubstantial changes from the claimed subject matter as viewed by a person with ordinary skill in the art, now known or later devised, are expressly contemplated as being equivalent within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be obviously substituted and also what essentially incorporates the essential idea of the invention. In addition, where the specification and claims refer to at least one of something selected from the group consisting of A, B, C . . . and N, the text should be interpreted as requiring only one element from the group, not A plus N, or B plus N, etc.

What is claimed is:

1. A contraceptive device-incorporated garment to promote a marketing, educational or social agenda, said contraceptive device-incorporated garment comprising:
a garment having an outside and an inside, a front side and a back side, wherein the garment is a T-shirt;
a contraceptive device coupled to the T-shirt, wherein the contraceptive device is a colored condom in a sealed package, and wherein the sealed package has at least one transparent side such that a color of the condom is visibly shown through the transparent side;
a pocket attached to and disposed on the front side and outside of the T-shirt via stitches, wherein the pocket has a top opening allowing insertion of the condom with its sealed package, and an outside of the pocket is measured 7.0 cm in height, and 7.0 cm in width, and has a circular transparent window having a diameter between 3.5 cm to 4.0 cm;
wherein the pocket has a compartment having a width between 6.0 cm to 6.5 cm;
wherein the condom is disposed within the compartment with said at least one transparent side facing an outside, and the color of the condom is visible through the window of the pocket;
a graphical display on the front side and outside of the T-shirt, wherein the graphical display has said color, and said condom as shown through the pocket window partially overlays over the said graphical display, and said color and said condom is incorporated as a part of a design of the graphical display;
a message element on the T-shirt, and is disposed between the outside of the T-shirt and the pocket, such that the message element is publicly visible through said pocket window when the condom is removed from the T-shirt, and thus an overall graphical display transpires a first message; and
wherein when the condom is inside the compartment, the overall graphical display transpires a second message.

2. The garment as recited in claim 2 wherein the sealed package of the condom has two transparent sides, such that when disposed within the pocket, the message element on the T-shirt is publicly visible and is shown through the colored condom and through the pocket window, and wherein the diameter of the window is no greater than 3.8 cm.

3. The garment as recited in claim 3 wherein the opening of the pocket is also sealed by stitches such that the condom is sealed inside the compartment, and removal of the condom requires removal of the stitches to the opening.
